(19) United States
(12) Patent Application Publication
(10) Pub. No.: US 2007/0032403 A1
(43) Pub. Date: Feb. 8, 2007

(54) Crystal structure of the ITK kinase domain

(75) Inventors: Joerg Martin Bentzien, White Plains, NY (US); Bennett Farmer II, Ridgefield, CT (US); Steven S. Pullen, Danbury, CT (US); Joey Studts, Ridgefield, CT (US); Andre White, Newtown, CT (US)

Correspondence Address: MICHAEL P. MORRIS;BOEHRINGER INGELHEIM CORPORATION, 900 RIDGEBURY ROAD, P O BOX 368, RIDGEFIELD, CT, 06877-0368, US

(73) Assignee: Boehringer Ingelheim Pharmaceuticals, Inc., Ridgefield, CT (US)

(21) Appl. No.: 11/021,759

(22) Filed: Dec. 23, 2004

Related U.S. Application Data
(60) Provisional application No. 60/533,434, filed on Dec. 30, 2003.

(51) Int. Cl. G06F 19/00; G01N 33/48; G01N 33/50; C12N 9/12; A61K 38/52

(52) U.S. Cl. 514/2; 435/194; 702/19

(57) ABSTRACT

Disclosed are polypeptides encoding the ITK kinase domain and nucleic acids encoding such polypeptides, crystal structures of various polypeptide-ligand complexes comprising the ITK kinase domain bound to a ligand, methods of producing the aforementioned polypeptides and nucleic acids which encode them and methods of producing crystal structures of the aforementioned polypeptides comprising the ITK kinase domain bound to a ligand.

### APPLICATION DATA

This application claims benefit to U.S. provisional No. 60/533,434 filed Dec. 30, 2003.

### FIELD OF INVENTION

The field of the invention relates to kinases, particularly ITK, which are attractive targets for the treatment of human diseases.

### BACKGROUND OF THE INVENTION

Kinases are key regulatory enzymes in eukaryotic signaling pathways. As such, kinases are attractive targets for pharmaceutical intervention in the treatment of human diseases. Non-receptor tyrosine kinases are critically involved in transmitting signals through antigen receptors on hematopoietic cells. Whereas the Src family and ZAP-70/Syk kinases function as on/off switches downstream of antigen receptors, the Tec family of kinases plays a signal amplification role (August et al., 2002, Int J Biochem Cell Biol 34:1184-1189).

Interleukin-2-inducible T cell kinase (ITK), also known as T cell-specific kinase (TSK) and expressed mainly in T cells (EMT) (Siliciano et al., 1992, Proc. Natl Acad. Sci. USA 89:11194-11198; Gibson et al., 1993, Blood 82:1561-1572; Heyeck and Berg, 1993, Proc. Natl. Acad. Sci. USA 90:669-673), is a member of the Tec kinase family whose expression is restricted to T cells, mast cells, and NK cells. ITK has been demonstrated to be involved in signaling through the T cell receptor (TCR) (reviewed in (Miller and Berg, 2002, Curr. Opin. Immunol. 14:331-340)) and, on mast cells, the high affinity IgE receptor (FcεRI) (Kawakami et al., 1995, J. Immunol. 155:3556-3562). Upon receptor cross-linking, upstream activation of Src family and ZAP-70/Syk kinases is required for activation of ITK. Src kinases phosphorylate ITK on the activation loop which is required before ITK can autophosphorylate leading to further activation (Heyeck et al., 1997, J Biol Chem 272:25401-25408). Additionally required for full activity, ITK must be recruited from the cytosol to the membrane through interactions with phosphatidyl inositol 3,4,5-trisphosphate produced upon PI3K activation and the SLP-76/LAT complex which is phosphorylated by ZAP-70/Syk kinases. These interactions are mediated by the ITK pleckstrin homology and the SH2 domains, respectively. Although numerous binding partners for ITK have been identified, the best understood role for ITK is in the phosphorylation of PLC-γ which is required for the production of inositol 1,4,5-trisphosphate and diacylglycerol which are necessary for calcium mobilization and PKC activation, respectively, thus activating numerous downstream pathways (reviewed in (August et al., 2002, Int J Biochem Cell Biol 34:1184-1189)).

In vivo studies on ITK have focused on its role in T cell development and function. In the absence of ITK, mice have 50% fewer CD4⁺ T cells due to a defect in positive selection. The surviving CD4⁺ T cells are defective in proliferation and cytokine production upon TCR stimulation in vitro or ex vivo. In vivo, ITK deficient mice do not mount a Th2 response to the pathogens Leishmania major, Nippostrongylus brasiliensis, or Schistosoma mansoni in contrast to wild-type mice (Liao and Littman, 1995, Immunity 3:757-769; Fowell et al., 1999, Immunity 11:399-409; Schaeffer et al., 2001, Nature Immunol. 2:1183-1188). Consistent with a defective Th2 response, ITK deficient mice exhibit reduced lung inflammation, eosinophil infiltration, and mucus secretion in an allergic asthma model (Mueller and August, 2003, J. Immunol. 170:5056-5063). Additional studies are still required to address the role of ITK in Th1 and CD8⁺ T cells in addition to mast cells in animal models of disease.

The catalytic domain of kinases contains conserved motifs that are required for protein structure and function. However, the precise tertiary structure of kinases, especially when bound by ligand, often cannot be predicted or modeled accurately. To date, three-dimensional structural data on the ITK kinase domain has not been available, thus hindering rational, structure-based design of antagonists to ITK kinase activity.

### SUMMARY OF THE INVENTION

It is therefore an object of the invention to provide both polypeptides encoding the ITK kinase domain and nucleic acids encoding such polypeptides.

It is another object of the invention to provide crystal structures of various polypeptide-ligand complexes comprising the ITK kinase domain bound to a ligand which provides the proper crystal structure as defined herein below.

It is yet a further object of the invention to provide methods of producing the aforementioned polypeptides and nucleic acids which encode them.

It is yet another object of the invention to provide methods of producing crystal structures of the aforementioned polypeptides comprising the ITK kinase domain bound to a ligand.

### BRIEF DESCRIPTION OF THE DRAWINGS

FIG. 1: Ribbon diagram representation of ITK/KD/G354:Compound 4 co-crystal structure. Secondary-structure elements are shown as arrows for beta-strands and as helices for alpha-helices. Compound 4 is shown with spheres for each non-hydrogen atom.

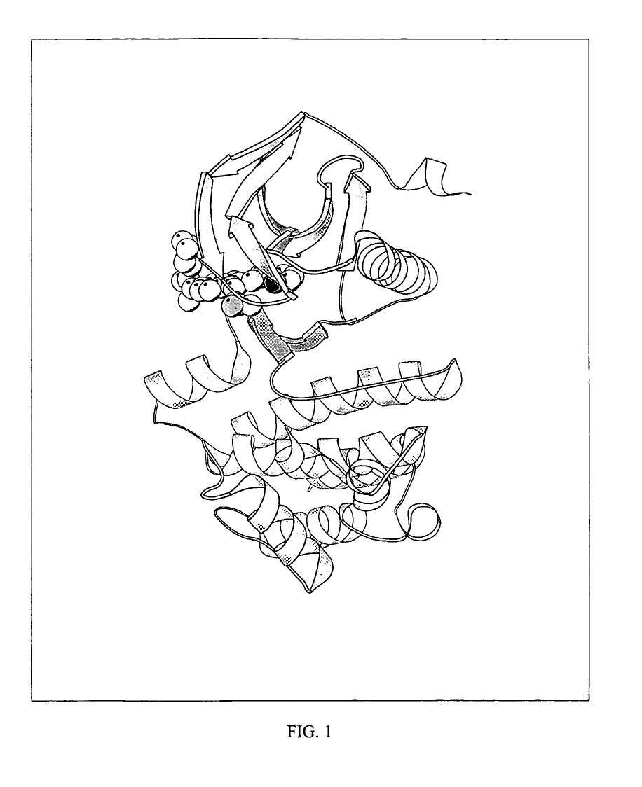

FIG. 2: Carbon-alpha trace of ITK/KD/G354 shown in stereo projection.

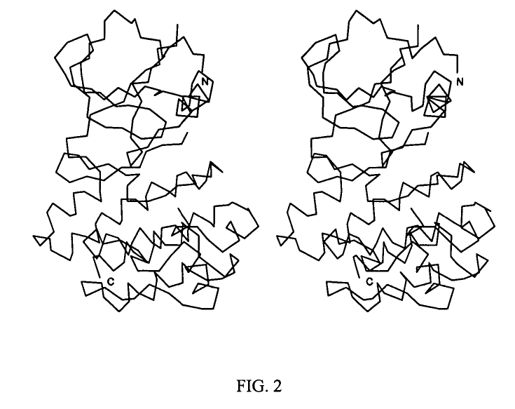

FIG. 3: Section of the electron density representation of Compound 4, shown in stereo projection. This electron density map is drawn with coefficients 2F_{obs}-F_{calc} and contoured at the level of the standard deviation of the entire map.

FIG. 4: Schematic representation of Compound 4 interactions with ITK/KD/G354. Hydrogen bonds are depicted with thick dash lines. Only Van der Waals interactions, i.e. only inter-molecular distances less than 3.8 Å between non-hydrogen atoms, are shown with dotted lines.

FIG. 5: Sequence alignment of the kinase domain from human, rat, mouse and zebra fish ITK orthologs.

### DESCRIPTION OF THE SEQUENCES

SEQ ID NO. 1 is the amino acid sequence of the human ITK kinase domain fragment ITK/KD/G354 used for X-ray crystallographic studies, which comprises ITK residues 354-620 of the full-length, wild-type ITK kinase (SEQ ID NO. 2). Thrombin cleavage of GST-ITK/KD/G354 (SEQ ID NO. 7) produces this kinase domain fragment which contains the vector-encoded sequence glycine-serine-methionine immediately N-terminal to ITK residue glycine 354.

SEQ ID NO. 2 is the amino acid sequence of the full-length, wild-type human ITK kinase (GenBank Accession No. AAQ02517).

SEQ ID NO. 3 is the amino acid sequence of the human ITK kinase domain fragment comprising ITK residues 343-620 of the full-length, wild-type ITK kinase, with an additional methionine encoded within the plasmid pITK/KD/Q343/GemT inserted immediately N-terminal to ITK residue glutamine 343.

SEQ ID NO. 4 is the DNA sequence of the plasmid pGST/1393 which is a baculoviral transplacement vector derived from pVL1393 (InVitrogen Life Technologies) which is modified to contain a gene encoding glutathione-S-transferase (GST) positioned 3′ to the polyhedrin promoter. DNA fragments inserted 3′ to the GST gene in the multiple cloning site produce fusion proteins C-terminal to GST upon expression.

SEQ ID NO. 5 is the amino acid sequence of the GST fusion protein to the human ITK kinase domain fragment comprising ITK residues 343-620 (GST-ITK/KD/Q343) of the full-length, wild-type ITK kinase. This GST fusion protein is encoded by the plasmid pGST-ITK/KD/Q343/1393.

SEQ ID NO. 6 is the amino acid sequence of the human ITK kinase domain fragment comprising ITK residues 354-620 of the full-length, wild-type ITK kinase, with an additional methionine encoded within the plasmid pITK/KD/G354/GemT inserted immediately N-terminal to ITK residue glycine 354.

SEQ ID NO. 7 is the amino acid sequence of the GST fusion protein to the human ITK kinase domain fragment comprising ITK residues 354-620 (GST-ITK/KD/G354) of the full-length, wild-type ITK kinase. This GST fusion protein is encoded by the plasmid pGST-ITK/KD/G354/1393.

SEQ ID NO. 8 is the amino acid sequence of the GST fusion protein to the human ITK kinase domain fragment comprising ITK residues 354-620 (GST-ITK/KD/G354) of the full-length ITK kinase in which phenylalanine residue 437 (numbered based on its position in the full-length, wild-type human ITK kinase) is substituted with a tyrosine residue (designated GST-ITK/KD/G354/F437Y). This GST fusion protein is encoded by the plasmid pGST-ITK/KD/G354/F437Y/1393.

SEQ ID NO. 9 is the amino acid sequence of the human ITK kinase domain fragment comprising ITK residues 343-620 of the full-length, wild-type ITK kinase (SEQ ID NO. 2). Thrombin cleavage of GST-ITK/KD/Q343 (SEQ ID NO. 5) produces this kinase domain fragment which contains the vector-encoded sequence glycine-serine-methionine immediately N-terminal to ITK residue glutamine 343.

SEQ ID NO. 10 is the amino acid sequence of the human ITK kinase domain fragment comprising ITK residues 361-620 of the full-length, wild-type ITK kinase, with an additional methionine encoded within the plasmid pITK/KD/S361/GemT inserted immediately N-terminal to ITK residue serine 361.

SEQ ID NO. 11 is the amino acid sequence of the GST fusion protein to the human ITK kinase domain fragment comprising ITK residues 361-620 (GST-ITK/KD/S361) of the full-length, wild-type ITK kinase. This GST fusion protein is encoded by the plasmid pGST-ITK/KD/S361/1393.

SEQ ID NO. 12 is the amino acid sequence of the human ITK kinase domain fragment comprising ITK residues 361-620 of the full-length, wild-type ITK kinase (SEQ ID NO. 2). Thrombin cleavage of GST-ITK/KD/S361 (SEQ ID NO. 11) produces this kinase domain fragment which contains the vector-encoded sequence glycine-serine-methionine immediately N-terminal to ITK residue serine 361.

SEQ ID NO. 13 is the amino acid sequence of the full-length, wild-type murine ITK kinase (GenBank Accession No. CA124846).

SEQ ID NO. 14 is the amino acid sequence of the murine ITK kinase domain fragment comprising ITK residues 353-619 of the full-length, wild-type murine ITK kinase, with an additional methionine encoded within the plasmid pmITK/KD/G353/TOPO inserted immediately N-terminal to ITK residue glycine 353.

SEQ ID NO. 15 is the amino acid sequence of the GST fusion protein to the murine ITK kinase domain fragment comprising ITK residues 353-619 (GST-mITK/KD/G353) of the full-length, wild-type ITK kinase. This GST fusion protein is encoded by the plasmid pGST-mITK/KD/G353/1393.

SEQ ID NO. 16 is the amino acid sequence of the murine ITK kinase domain fragment comprising ITK residues 353-619 of the full-length, wild-type murine ITK kinase (SEQ ID NO. 13). Thrombin cleavage of GST-mITK/KD/G353 (SEQ ID NO. 15) produces this kinase domain fragment which contains the vector-encoded sequence glycine-serine-methionine immediately N-terminal to ITK residue glycine 353.

### DETAILED DESCRIPTION OF THE INVENTION

This invention provides certain crystallized, protein kinase-ligand complexes, in particular ITK kinase domain-ligand complexes, and their structural coordinates. The structural coordinates are based on the structure of a ligand-bound ITK kinase domain complex that has now been solved and refined to a resolution 3.0 Å and which reveals new structural information. The key structural features of the ITK kinase domain, particularly the shape of the ATP-binding site, are useful to methods for designing inhibitors of the ITK kinase activity and for solving the structures of other proteins with similar features.

In one embodiment, the invention provides a crystal of a polypeptide-ligand complex that comprises the ITK kinase domain and a ligand. Preferred ITK kinase domains include ITK/KD/S361 (SEQ ID NO. 12), ITK/KD/Q343 (SEQ ID NO. 9) and ITK/KD/G354 SEQ ID NO. 1, with the most preferred being human ITK/KD/G354.

In another embodiment, the invention provides a crystal of a polypeptide-ligand complex that comprises the ITK kinase domain ITK/KD/G353 SEQ ID NO. 16.

It shall be understood that all ITK kinase domains described herein are mammalian, preferably human and murine. In describing protein structure and function, reference is made to amino acids comprising the protein. It shall be understood that the terms protein and polypeptide can be used interchangeably and are both defined as a polymer of two or more amino acids covalently linked by peptide bonds. The amino acids may also be referred to by their conventional abbreviations, as shown in the Table 1 below.

TABLE 1

| | |
|---|---|
| A = Ala = Alanine | T = Thr = Threonine |
| V = Val = Valine | C = Cys = Cysteine |
| L = Leu = Leucine | Y = Tyr = Tyrosine |
| I = Ile = Isoleucine | N = Asn = Asparagine |
| P = Pro = Proline | Q = Gln = Glutamine |
| F = Phe = Phenylalanine | D = Asp = Aspartic Acid |
| W = Trp = Tryptophan | E = Glu = Glutamic Acid |
| M = Met = Methionine | K = Lys = Lysine |
| G = Gly = Glycine | R = Arg = Arginine |
| S = Ser = Serine | H = His = Histidine |

A crystal, as defined herein below, according to the invention may take a variety of forms, all of which are included in the present invention.

The term ‘ligand’ shall be understood to include any molecule that forms a complex with an ITK kinase domain, as defined herein below, according to the invention and can be used to form a crystal of the present invention. Preferred ligands include substituted benzimidazole compounds shown in Table 2 below. Analogs, positional and stereoisomer isomers thereof which provide a crystal structure are within the scope of the invention and will be apparent to those of ordinary skill in the art.

### Isolating the ITK Kinase Domain

DNA Cloning and Baculovirus Generation

In one aspect of the invention, there is provided novel nucleic acids encoding the ITK kinase domain as described herein below. In yet another aspect of the invention, there is provided vectors comprising said nucleic acids. The nucleic acids and vectors are prepared as follows:

A DNA fragment encoding amino acids 343-620 of the full-length, wild-type human ITK kinase (SEQ ID NO. 2) was PCR-amplified from an unstimulated human peripheral blood leukocyte cDNA library (Clontech) using oligonucleotide pairs 5′-GGGATCCATGCAGAAAGCCCCAGTTACAGCAGG-3′ and 5′-GCGGCCGCCTAAAGTCCTGATTCTGCAATTTCAGCC-3′ and ligated into pGem-T (Promega) to make pITK/KD/Q343/GemT wherein a methionine residue is inserted immediately N-terminal to Q343 of ITK to generate the predicted ITK kinase domain protein in SEQ ID NO. 3. The BamHI to NotI ITK kinase domain encoding fragment from pITK/KD/Q343/GemT was ligated into pGST/1393 (SEQ ID NO. 4) at the same sites to generate pGST-ITK/KD/Q343/1393 which encodes a GST-ITK/KD/Q343 fusion protein (SEQ ID NO. 5). A DNA fragment encoding amino acids 354-620 of the full-length, wild-type human ITK kinase (SEQ ID NO. 2) was PCR-amplified from pGST-ITK/KD/Q343/1393 using oligonucleotide pairs 5′-GGGATCCATGGGGAAATGGGTGATCGACC-3′ and 5′-GCGGCCGCCTAAAGTCCTGATTCTGCAATTTCAGCC-3′ and ligated into pGem-T to make pITK/KD/G354/GemT wherein a methionine residue is inserted immediately N-terminal to G354 of ITK to generate the predicted ITK kinase domain protein in SEQ ID NO. 6. A DNA fragment encoding amino acids 361-620 of the full-length, wild-type human ITK kinase was PCR-amplified from pGST-ITK/KD/Q343/1393 using oligonucleotide pairs 5′-GGGATCCATGTCAGAGCTCACTTTTGTGC-3′ and 5′-GCGGCCGCCTAAAGTCCTGATTCTGCAATTTCAGCC-3′ and ligated into pGem-T to make pITK/KD/S361/GemT wherein a methionine residue is inserted immediately N-terminal to S361 of ITK to generate the predicted ITK kinase domain protein in SEQ ID NO. 10. The BamHI to NotI ITK kinase domain encoding fragments from pITK/KD/G354/GemT and pITK/KD/S361/GemT were ligated into pGST/1393 at the same sites to generate pGST-ITK/KD/G354/1393 and pGST-ITK/KD/S361/1393 which encode the fusion proteins GST-ITK/KD/G354 (SEQ ID NO. 7) and GST-ITK/KD/S361 (SEQ ID NO. 11), respectively. pGST-ITK/KD/G354/F437Y/1393 which, in the GST-ITK/KD/G354 construct, encodes a tyrosine in place of a phenylalanine at residue 437 of the full-length human ITK kinase sequence was generated using the complementary oligonucleotides 5′-CCTGGTGTTTGAGTACATGGAGCACGGCT-3′ and 5′-AGCCGTGCTCCATGTACTCAAACACCAGG-3′ and using pGST-ITK/KD/G354/1393 as a template with the QuikChange site-directed mutagenesis kit (Stratagene) to generate pGST-ITK/G354/F437Y/1393 which encodes GST-ITK/G354/F437Y (SEQ ID NO. 8). A DNA fragment encoding amino acids 342-619 from the murine ITK kinase (SEQ ID NO. 13) was PCR-amplified from a mouse spleen cDNA library (Clontech) using oligonucleotide pairs 5′-CAAAAAGCCCCTGTCAC-3′ and 5′-GGCGGCCGCCTAAAGCCCAGCTTCTGCG-3′ and ligated into TrcHis2-TOPO (Invitrogen) to make pmITK/KD/Q342/TOPO. A DNA fragment encoding amino acids 353-619 from the murine ITK kinase (SEQ ID NO. 13) was PCR-amplified from pmITK/KD/Q342/TOPO using oligonucleotide pairs 5′-GGGATCCATGGGGAAGTGGGTGATCCAAC-3′ and 5′-GGCGGCCGCCTAAAGCCCAGCTTCTGCG-3′ and ligated into pCRII-TOPO (Invitrogen) to make pmITK/KD/G353/TOPO wherein a methionine residue is inserted immediately N-terminal to G353 of ITK to generate the predicted protein in SEQ ID NO. 14. The BamHI to NotI ITK kinase domain encoding fragment from pmITK/KD/G353/TOPO was ligated into pGST/1393 (SEQ ID NO. 4) at the same sites to generate pGST-mITK/KD/G353/1393 which encodes a GST-mITK/KD/G353 fusion protein (SEQ ID NO. 15). Recombinant baculovirus stocks were generated by standard methods (O'Reilly et al., 1992, Baculovirus Expression Vectors: A Laboratory Manual, W.H. Freeman & Co.) using the pGST-ITK/KD/Q343/1393, pGST-ITK/KD/G354/1393, pGST-ITK/G354/F437Y/1393, pGST-ITK/KD/S361/1393, pGST-mITK/KD/G353/1393 vectors.

In yet another aspect of the invention, there is provided a process of producing the ITK kinase domain polypeptides as described herein. Said polypeptides can be produced as follows:

Protein Expression and Purification

Spodoptera frugiperda (Sf21) cells were maintained and infected as described previously (Dracheva et al., 1995, J Biol Chem 270:14148-14153) using medium supplemented with 5% heat-inactivated fetal bovine serum (Hyclone) and 50 μg/ml gentamicin sulfate (Life Technologies, Inc.). All purification procedures were performed at 4° C. Cytosolic extracts of baculovirus-infected Sf21 cells were prepared as described (Pullen et al., 1998, Biochemistry 37:11836-11845). Extracts were applied to a glutathione sepharose 4B column (Amersham) equilibrated in Buffer A (20 mM HEPES, pH 7.5, 150 mM NaCl, 1 mM TCEP, 10% v/v glycerol, 0.1 mM EDTA, 0.1 mM EGTA, and 1 mM PMSF). The column was washed with Buffer A containing 400 mM NaCl. GST-ITK/KD/G354 and GST-ITK/KD/G354/F437Y were eluted in Buffer A containing 150 mM NaCl and 10 mM glutathione. Alternatively, protein was eluted by flowing bovine thrombin (USB) at 20 units/mL onto the column in 50 mM Tris, pH 8.0, 2.5 mM CaCl₂, 10% v/v glycerol, and 150 mM NaCl. The amino acid sequences of the resulting thrombin cleaved proteins ITK/KD/Q343, ITK/KD/G354, ITK/KD/S361, and mITK/KD/G353 are shown in SEQ ID NO. 9, SEQ ID NO. 1, SEQ ID NO. 12, and SEQ ID NO. 16, respectively. Peak fractions containing the ITK kinase domain were pooled, diluted with an equal volume of Buffer B (10 mM Tris, pH 7.2, 100 mM NaCl, 5% glycerol, 0.5 mM TCEP), applied to a Macro-Prep DEAE column equilibrated in Buffer B, and proteins were eluted with a 0 to 500 mM NaCl gradient in Buffer B. Peak fractions were pooled and applied to a Sephacryl S-100 HR column preequilibrated with 10 mM HEPES, pH 7.5, 100 mM NaCl, and 1.0 mM TCEP. Peak fractions were pooled, concentrated to approximately 30 mg/ml using a Vivaspin 30 K MWCO concentrator (Sartorius), quantified, frozen in aliquots under liquid nitrogen, and stored at −80° C. Sample purity was verified by SDS-PAGE analysis and electrospray ionization mass spectrometry.

Definition of Kinase Domain Fragment

The term ITK kinase domain shall be understood to mean a polypeptide construct comprising residues 354-620 of human ITK (ITK/KD/G354) which binds a ligand as defined herein. Such bound ligands include, but are not limited to, inhibitor small molecules. This inhibition is expressed as an IC₅₀ value that is determined either by the Tec Family Kinase Assay described below or by other methods known in the art to measure μM to nM IC₅₀ values for small-molecule inhibitors. Examples of small molecule ITK inhibitors include, but are not limited to, those compounds shown in Table 2 and Compound 8. ITK/KD/Q343 was subjected to limited digestion with trypsin to identify fragments of the ITK kinase domain that are resistant to proteolysis due to being folded in a more stable and compact conformation. Protein fragments were identified by matrix-assisted laser desorption/ionization time-of-flight mass spectrometry analysis. Characterization of the fragments indicated that residues N-terminal to R352 are accessible to trypsin. Consequently, an ITK kinase domain construct including residues 354-620 of human ITK (ITK/KD/G354) was designed to increase the stability of the protein. Additionally, an ITK kinase domain construct including residues 361-620 of human ITK (ITK/KD/S361) was designed to remove all but two residues N-terminal to subdomain I of the kinase, based on the Hanks classification of protein kinases (Hanks and Hunter, 1995, FASEB J. 9:576-596), in case these are disordered in the context of the shorter ITK/KD/G354 construct. These constructs are described in the DNA cloning and baculovirus generation section.

Selection of Murine ITK Kinase Domain Fragment

The murine ITK kinase domain fragment including residues 342-619 has 3 conservative and 7 non-conservative amino acid substitutions when compared with human ITK kinase domain residues 343-620. To improve the likelihood of obtaining ITK kinase domain protein crystals of suitable size and quality, an ITK kinase domain construct including residues 353-619 of murine ITK (mITK/KD/G353) homologous to the stable human fragment (ITK/KD/G354) was designed as described in the DNA cloning and baculovirus generation section.

Tec Family Kinase Assay

ITK is purified as a GST-fusion protein to test for catalytic activity. The kinase activity is measured using DELFIA (Dissociation Enhanced Lanthanide Fluoroimmunoassay) which utilizes europium chelate-labeled anti-phosphotyrosine antibodies to detect phosphate transfer to a random polymer, poly Glu₄: Tyr₁ (PGTYR). The screen utilizes the Zymark Allegro UHTS system to dispense reagents, buffers and samples for assay, and also to wash plates. The kinase assay is performed in kinase assay buffer (50 mM HEPES, pH 7.0, 25 mM MgCl₂, 5 mM MnCl₂, 50 mM KCl, 100 μM Na₃VO₄, 0.2% BSA, 0.01% CHAPS, 200 μM TCEP). Test samples initially dissolved in DMSO at 1 mg/mL, are pre-diluted for dose response (9 doses with starting final concentration of 3 μg/mL, 1 to 3 serial dilutions) with the assay buffer in 384-well polypropylene microtiter plates. A 10 μL volume/well of a mixture of substrates containing 15 μM ATP and 9 ng/μL PGTYR-biotin (CIS Biointernational) in kinase buffer is added to neutravidin coated 384-well white plate (PIERCE), followed by 20 μL/well test sample solution and 20 μL/well of diluted enzyme (1-7 nM final concentration). Background wells are incubated with buffer, rather than 20 μL enzyme. The assay plates are incubated for 30 min at room temperature. Following incubation, the assay plates are washed three times with 100 μL wash buffer (50 mM Tris-HCL, pH 7.4, 150 mM NaCl, 0.05% Tween 20, 0.2% BSA). A 50 μL aliquot of europium-labeled anti-phosphotyrosine (Eu³⁺-PT66, Wallac CR04-100) diluted in 50 mM Tris-HCl, pH 7.8, 150 mM NaCl, 10 μM DTPA, 0.05% Tween 40, 0.2% BSA, 0.05% BGG (1 nM final concentration) is added to each well and incubated for 30 min at room temperature. Upon completion of the incubation, the plate is washed four times with 100 μL of wash buffer and 50 μL of DELFIA Enhancement Solution (Wallac) is added to each well. After 15 min, time-resolved fluorescence is measured on the LJL's Analyst (excitation at 360 nm, emission at 620 nm, EU 400 Dichroic Mirror) after a delay time of 250 μs. An IC₅₀ can be obtained by fitting the rates vs. compound/ligand concentration data into a simple competitive inhibitor model. Under these assay conditions, a 3-fold difference in compound potency (IC₅₀) is considered within the variation of the assay. Preferred ligands will have an IC₅₀<1000 nM.

6. Thermal Denaturation of ITK Constructs

Thermal denaturation experiments were performed on a Jasco J-720 spectropolarimeter equipped with a Peltier thermostatic cell holder. For each measurement, a 1 cm quartz cuvette was loaded with 5 μM ITK kinase domain construct in a pH 7.0 buffer containing 10 mM sodium phosphate, 100 mM NaCl and 1 mM TCEP. Absorbance data at 230 nm was collected as the temperature was scanned from 2 to 100° C. at a ramp rate of 0.2° C./min. The melting temperature (Tₘ) for each sample was calculated as the maximum deflection point of the first derivative of the melting transition using Origin (version 7.0).

Rational Mutant Design ITK/KD/G354 F437Y

The ITK residues Q367, I369, L379, K387, and F437, numbered based on the position in the full-length, wild-type human ITK kinase, define a shallow, surface-exposed hydrophobic pocket on ITK/KD/G354 that has not been described in previous kinase crystal structures. The observed interaction of Compound 4 with this hydrophobic pocket region suggests that it could contribute to favorable compound interaction. Moveover, Phe437 extends its side chain toward Compound 4 such that the Phe Cζ (4-position aromatic carbon) is found in close proximity to the cyclohexyl moiety of Compound 4. These observations suggest that an additional hydroxyl on this protein Phe437 residue side chain, such as that found in a Phe437Tyr mutant, may be less favorable for the binding of compounds having a hydrophobic six-membered ring at the Compound 4 cyclohexyl position. To test this hypothesis, a human ITK mutant has been generated where Phe437 is mutated to a tyrosine residue (F437Y mutant) in the construct GST-ITK/KD/G354 to generate GST-ITK/KD/G354/F437Y. Tyrosine is the most common amino acid at this position of kinases (Kostich et al., 2002, Genome Biol. 3:1-12) and would be predicted to interfere with the binding of Compound 4 based both on the introduction of a polar functionality into the hydrophobic pocket and on a steric interaction with the cyclohexyl ring of Compound 4. As indicated in Table 2, ITK with the F437Y substitution (GST-ITK/KD/G354/Y437Y) is inhibited less than the wild-type ITK kinase domain (GST-ITK/KD/G354) by compounds with a six-membered hydrophobic ring functionality, such as an optionally substituted aryl, heteroaryl or cycloalkyl moiety. In contrast, compounds lacking this hydrophobic functionality have similar potency against the wild-type and F437Y substituted kinase. These results demonstrate that compounds including such a hydrophobic functionality selectively inhibit ITK which contains the shallow, hydrophobic pocket versus kinases with a tyrosine at the analogous position to ITK residue 437 in which this pocket is perturbed. These findings are consistent with: (1) the shallow hydrophobic pocket contributing to binding interactions with the compounds and thus providing a previously undescribed site for interactions with kinase inhibitors; (2) residue 437 being involved in compound specificity in a protein containing a tyrosine at this position, such as the Phe437Tyr ITK/KD/G354 mutant.

TABLE 2

| | | | ITK | Fold |
|---|---|---|---|---|
| | | ITK | F437Y | Difference |
| Compound | Structure | IC₅₀ (nM) | IC₅₀ (nM) | in IC₅₀ |
| 1 | 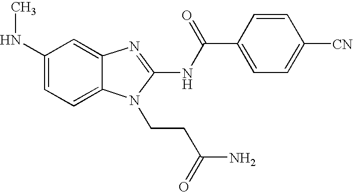 | 460 | 1200 | 2.6 |
| 2 | 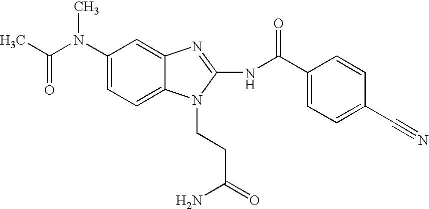 | 170 | 250 | 1.5 |
| 3 | 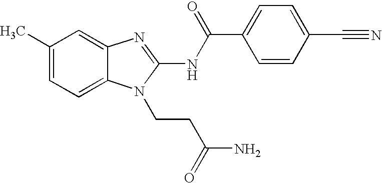 | 440 | 500 | 1.1 |
| 4 | 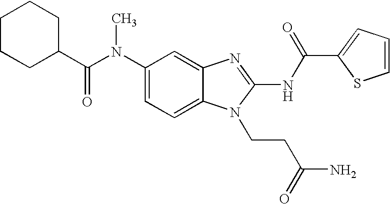 | 110 | 830 | 7.5 |
| 5 | 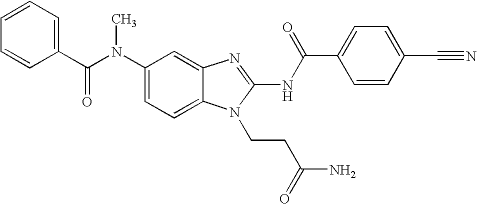 | 29 | 480 | 16.6 |
| 6 | 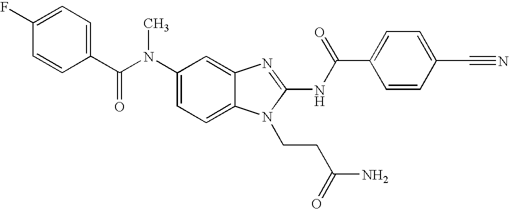 | 9 | 170 | 18.9 |
| 7 | 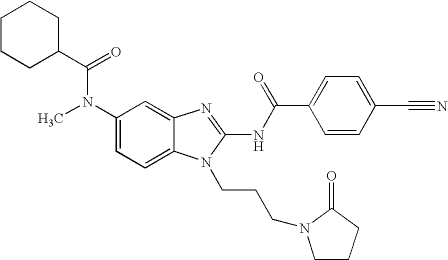 | 15 | 160 | 10.7 |

ITK/KD/G354 Crystallization

The term ‘crystal’ or ‘protein crystal’ shall be understood to mean a product of the process of obtaining crystals of the ITK kinase domain protein-ligand complex, with said process comprising:

(a) obtaining a crystallizable composition, with said crystallizable composition comprising an ITK kinase domain protein, suitable cations and a ligand according to the invention; and

(b) subjecting the composition of step (a) to conditions which promote crystallization.

Another aspect of this invention relates to a method for preparing crystals containing an ITK kinase domain protein-ligand complex. It is inferred by those skilled in the art that a variety of techniques and suitable conditions which promote crystallization may be used to grow protein or protein-ligand crystals. This includes, but is not limited to, batch, under-oil batch, dialysis, vapor diffusion by either sitting or hanging drops, and liquid bridge (Ducruix and Geige, 1992, Crystallization of Nucleic Acids and Proteins: A practical Approach, IRL Press, Oxford, England; McPherson, 1999, Crystallization of Biological Macromolecules, Cold Spring Harbor Laboratory Press, New York). In the most general case, any one of the above techniques may be used to grow ITK/KD/G354 crystals. In a preferred embodiment, the vapor diffusion method is used to grow ITK/KD/G354 crystals. In a more preferred embodiment, Compound 4 is used to grow ITK/KD/G354 protein-ligand crystals by means of the vapor diffusion method. In an even more preferred embodiment, hanging drops are used with the vapor diffusion method and with Compound 4 to grow ITK/KD/G354 protein-ligand crystals. A most preferred crystallization protocol is disclosed in the examples section.

The vapor diffusion method involves the equilibration of one or more drops containing the protein formulation against a larger reservoir solution in a sealed well. These drops may be sitting or hanging. In the most general case, the reservoir solution contains a precipitant constituent that is more concentrated than it is in the drop. In a preferred embodiment, this precipitant is 5 to 15% polyethylene glycol 1500 (PEG 1500). In an even more preferred embodiment, 8 to 12% PEG 1500 is used. A most preferred crystallization protocol is disclosed in the examples section. Such a formulation is applicable with human ITK/KD/G354-ligand, but is different when used with the murine ITK/KD/G353-compound preparation. The preferred precipitant for murine ITK/KD/G353-compound crystallization is 7 to 30% polyethylene glycol 5000 monomethylether, a more preferred precipitant being 10 to 18% polyethylene glycol 5000 monomethylether. A most preferred crystallization protocol is disclosed in the examples section.

The crystallization solution pH is an important factor influencing protein crystallization. Commonly, an optimal pH is achieved by adjusting the reservoir solution pH and by using some of this solution in the protein crystallization drop. In a preferred embodiment, the reservoir solution contains 100 mM sodium citrate pH 5 to 6. In a more preferred embodiment, the buffer solution used is first adjusted to pH 5.2-5.4, then autoclaved, at which point the buffer pH changes to values of 5.55-5.75, and finally used with the other constituents to form the reservoir solution. A most preferred protocol is disclosed in the examples section.

Once a crystal of the present invention is grown, it can be characterized by X-ray diffraction. More than one method may be used to generate X-rays and to characterize the diffraction pattern. For example, X-rays used may be generated from a conventional source, such as a sealed tube or rotating anode, or from a synchrotron source. Methods of characterization include, but are not limited to, diffractometer data collection, precession photography and Laue diffraction. Data may be processed using D*TREK (Rigaku MSC), MOSFLM (Leslie, 1999, Acta Crystallogr. D 55:1696-1702) or a combination of DENZO and SCALEPACK (Otwinowski and Minor, 1997, Meth. Enzymol. 276:307-326). Examples herein provide a statistical sampling of X-ray diffraction data measurement, data reduction and analyses.

Crystal Structure Determination

A structure determination using X-ray diffraction requires phase angle estimates to be combined with the diffraction data. In the case of macromolecules, such phase angle estimates may be derived from a known structure of similar topology, from ab initio using indirect methods, or a combination thereof. The former is achieved using molecular replacement methods, where the unit cell molecular arrangement of the unknown structure is rebuilt computationally using the structure of a known molecule of assumed similar topologically. Examples of molecular replacement algorithms include AMoRe (Navaza, 1994, Acta Crystallogr. A 50:157-163), EPMR (Kissinger et al., 1999, Acta Crystallogr. D 55:484-491), MERLOT (Fitzgerald, 1988, J. Appl. Cryst. 21:273-278) and X-PLOR (Brünger et al., 1987, Science 235:458-460). Alternatively, phase angle estimates may be obtained indirectly. Examples of such methods include isomorphous replacement, single-isomorphous replacement with anomalous scattering, single wavelength anomalous dispersive and multiwavelength anomalous diffraction.

In a preferred embodiment, the structure of the protein-ligand complex specified as ITK/KD/G354-Compound 4 is determined by the method of molecular replacement. One aspect of this method is the molecular arrangement of the proteins forming the crystal. Most commonly found in known crystal structures are one, occasionally two, and rarely more than two protein molecules per asymmetric unit. Exemplified herein is a assembly of six individual ITK/KD/G354 protein molecules that define the asymmetric unit which also includes an estimated 72% solvent. These six molecules are replicated six times according to the hexagonal unit cell space group symmetry P6₄ to form the unit cell and subsequently replicated along axes a, b and c to form the crystal. ITK/KD/G354 crystals are thus formed from nanotubes of protein molecules with a solvent channel size of about 90 Å diameter.

The molecular replacement method inherently produces an initial set of atomic coordinates for the protein referred to as the structure. This structure is subjected to rounds of refinement interspersed with model rebuilding using 0 (Jones et al., 1991, Acta Crystallogr. A 47:110-119) or Quanta (Accelrys). Refinement is performed using CNX (Accelrys; Brünger, 1988, J. Mol. Biol. 203:803-816), REFMAC (Murshudov et al., 1997, Acta Crystallogr. D 53:240-255) or other protein refinement software. Refinement protocols used herein aim at improving the fit of the structure to the experimental data by minimizing the difference between the calculated amplitudes F_{calc}, which are generated from the structure, and the observed structure factor amplitudes F_{obs}, which are obtained from the experimental X-ray diffraction intensity data. Ideal stereochemical parameters (Engh and Huber, 1991, Acta Crystallogr. A 47:392-400) are used to incorporate expected standard geometry constraints in the refinement.

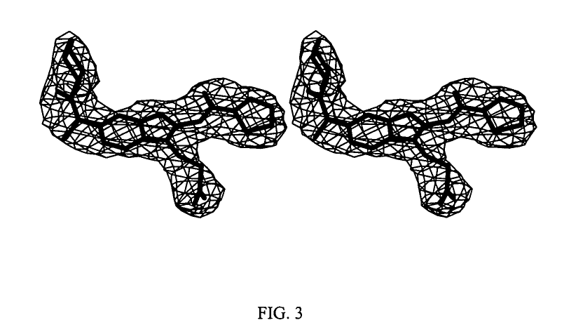

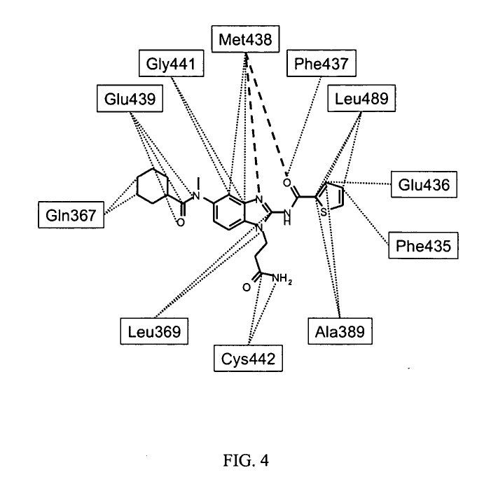

One aspect of the present invention is the ITK/KD/G354 binding pocket that accommodates Compounds 4 and 8. FIG. 3 shows the conformation that Compound 4 adopts when bound to ITK/KD/G354. The cyclohexyl moiety of Compound 4 is oriented nearly orthogonal to the benzimidazole scaffold. FIG. 4 illustrates the molecular interactions between ITK/KD/G354 and Compound 4. Among these interactions, the protein main chain of Met438 forms two hydrogen-bonds with Compound 4. Also noteworthy is the proximity between the cyclohexyl of Compound 4 and the Phe437 phenyl side chain: Some atoms of these two groups come as close as about 3.8 Å to each other. One possible implication of such proximity is compound selectivity.

Those of skill in the art understand that a set of structural coordinates for an enzyme, an enzyme-complex, or a portion thereof, is a relative set of points that define a shape in three dimensions. Thus, it is possible that an entirely different set of coordinates could define a similar or identical shape. Moreover, slight variations in the individual coordinates will have little effect on the overall shape. In terms of binding pockets, these variations would not be expected to significantly alter the nature of ligands that could associate with those pockets.

Modifications in the crystal structure due to mutations, additions, substitutions, and/or deletions of amino acids, or other changes in any of the components that make up the crystal could also account for variations in structural coordinates. If such variations are within an acceptable standard error as compared to the original coordinates, the resulting three-dimensional shape is considered to be the same. Thus, for example, a ligand that binds to the Compound 4-binding region of ITK kinase domain would also be expected to bind to another binding pocket whose structural coordinates defined a shape that fell within the acceptable error.

Still another aspect of the present invention comprises a method for using a protein crystal structure of the present invention in a drug screening assay. In one such embodiment, the method comprises identifying a compound as a potential inhibitor by performing rational drug design with a three-dimensional structure determined for the crystal, preferably in conjunction with computer modeling. Such computer modeling is preferably initiated with a program that incorporates a Docking algorithm (Dunbrack et al., 1997, Folding & Design 2:R27-42). Examples of such programs include DOCK (Kuntz et al., 1982, J. Mol. Biol. 161:269-288), GRID (Goodford, 1985, J. Med. Chem. 28:849-857), AUTODOCK (Goodsell and Olsen, 1990, Proteins. Struct., Funct., Genet. 8:195-202), MCSS (Miranker and Karplus, 1991, Proteins 11:29-34), GOLD (Jones et al., 1995, J Mol Biol 245:43-53), QXP (McMartin and Bohacek, 1997, J. Comput. Aided Molec. Des. 11:333-344), FlexE (Claussen et al., 2001, J Mol Biol 308:377-395), Glide (Shrodinger, Portland, Oreg.), FlexX (Sybl, Tripos, St. Louis, Mo.) and ICM (Molsoft, San Diego, Calif.; http://www.molsoft.com). With such programs, one or more compounds are each brought into contact with a binding site, in this case the ATP binding site, on the ITK kinase domain. The compound binding modes, of which there may be several for each compound and which both describe the translational and orientational relationships between the protein and that compound and also define the conformation of that compound, are then scored to provide a theoretical guide to the binding affinity of each compound for the particular binding site on the ITK kinase domain compound is selected as a potential inhibitor based on the scores assigned to its various binding modes to the ITK kinase domain.

In a preferred embodiment of this type, a supplemental crystal is grown which comprises a protein-ligand complex formed between an ITK kinase domain and an initial inhibitor. Preferably the crystal effectively diffracts X-rays such that the atomic coordinates of the protein-inhibitor complex can be determined to a resolution of better than 5.0 Angstroms, more preferably better than 3.0 Angstroms. The three-dimensional structure of the supplemental crystal is determined by molecular replacement analysis, multiwavelength anomalous dispersion, multiple isomorphous replacements, or a combination thereof. A new inhibitor with potentially greater binding affinity is then identified by structure-based design techniques using the three-dimensional structure determined for the supplemental crystal, preferably in conjunction with computer modeling. The potentially improved inhibitor is then tested in a protein kinase assay such as the Tec Family Kinase Assay described herein above.

All literature and patent references cited in this application are incorporated herein by reference in their entirety.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the invention.

### EXAMPLES

### Example 1

### ITK/KD/G354 Expressed Protein

A significant obstacle in the process of obtaining the 3-dimensional structure of a protein by X-ray crystallography is the identification of a protein fragment that is amenable to the formation of protein crystals of suitable quality. Since kinases often have multiple domains, one strategy that increases the likelihood of generating protein crystals of the kinase domain (catalytic) fragment has been to delete the other domains. Furthermore, by making a kinase domain fragment analogous to that of a related kinase for which a crystal structure has been determined, one may further increase the probability of generating suitable protein crystals. Using these concepts, the human ITK/KD/Q343 construct was generated. Theory predicts that a more compactly folded protein has a greater probability of producing high quality protein crystals. Therefore, the human ITK/KD/Q343 construct was subjected to limited proteolysis with trypsin to identify any fragments of the construct that are easily accessible to the protease and are thus likely to be less compactly folded. Using this strategy, the human ITK/KD/G354 and ITK/KD/S361 constructs were designed. To prioritize the three human ITK kinase domain protein fragments for crystallization trials, each was assayed for catalytic activity and for structural foldedness by circular dichroism-monitored thermal denaturation. Both ITK/KD/Q343 and ITK/KD/G354 showed significant catalytic activity, whereas ITK/KD/S361 did not possess activity above background levels (Table 3). In thermal denaturation studies, ITK/KD/G354 had a higher Tₘ than ITK/KD/Q343, suggesting that ITK/KD/G354 is a more stably folded protein. ITK/KD/S361 had the highest Tₘ, but the lack of catalytic activity suggests that this protein may not have a functionally competent catalytic site in the kinase domain. Consequently, the ITK/KD/G354 construct was prioritized for structural studies since it was catalytically active and predicted to be tightly folded. To further increase the probability of obtaining suitable crystals, a murine construct (ITK/KD/G353) analogous to the human ITK/KD/G354 construct was generated. This murine ITK kinase domain construct has 3 conservative and 7 non-conservative amino acid substitutions when compared with the human ITK/KD/G354 construct which may alter the conformation or surface properties of the protein thus providing alternative opportunities for crystal formation.

TABLE 3

| Kinase | Kinase Activity* | |
|---|---|---|
| Construct | (Fluorescent Units) | Tₘ (° C.) |
| ITK/KD/Q343 | 2000 | 50.0 |
| ITK/KD/G354 | 910 | 67.2 |
| ITK/KD/S361 | 30 | 72.8 |

*Using 250 nM enzyme.

### Example 2

### Preparation of ITK/KD/G354-Compound 4 Complex

Previously stored at −80° C., human ITK/KD/G354 protein at a concentration between 16 and 30 mg/mL is thawed on ice for 30 min before use. Compound 4, thiophene-2-carboxylic acid [1-(2-carbamoyl-ethyl)-5-(cyclohexanecarbonyl-methyl-amino)-1H-benzoimidazol-2-yl]-amide, is dissolved with dimethyl sulfoxide (DMSO) to a concentration of 50 to 100 mg/mL at room temperature. Protein solution is mixed with a 3 to 5 molar ratio of Compound 4 and incubated on ice for 60 to 90 min. The complex is then diluted to a protein concentration of about 16 mg/mL using an ice-cold solution of 10 to 20 mM 4-(2-hydroxyethyl)-1-piperazine-ethanesulfonic acid (HEPES) pH 7.5, 100 mM NaCl and 1 mM Tri(2-carboxyethyl)phosphine hydrochloride (TCEP).

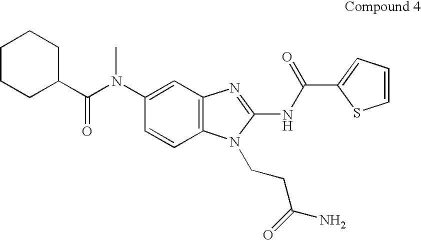

### Example 3

### ITK/KD/G354 Protein Crystallization

Protein crystals are obtained by the vapor diffusion method using hanging drops (McPherson, 1999, Crystallization of Biological Macromolecules, Cold Spring Harbor Laboratory Press, New York) at 4° C. A drop containing the protein is let equilibrate against a reservoir solution in a sealed container. In the present example, a drop is prepared by mixing 2 μL of the ITK/KD/G354-Compound 4 complex with 1 μL of reservoir solution. The reservoir solution contains 5 to 15% PEG 1500, 100 mM sodium citrate pH 5 to 6 and 1 mM TCEP. Crystals typically appear between 3 to 5 weeks after setup and continue to grow to a typical size of 200 to 250 μm within 3 to 5 months.

Crystals are soaked with compound 8, N-{5-(cyclohexanecarbonyl-methyl-amino)-1-[3-(4-methyl-piperazin-1-yl)-propyl]-1H-benzoimidazol-2-yl}-4-iodo-benzamide, in the following way. Crystals are transferred to a stabilizing solution containing 35% PEG 1500, 100 mM sodium citrate pH 5.30, 1 mM TCEP and 5 mM Compound 8. Following an incubation period of 24-36 hrs, crystals are briefly transferred to that same solution containing an added 20% glycerol. The recovered crystals are then frozen in liquid nitrogen and kept frozen until use.

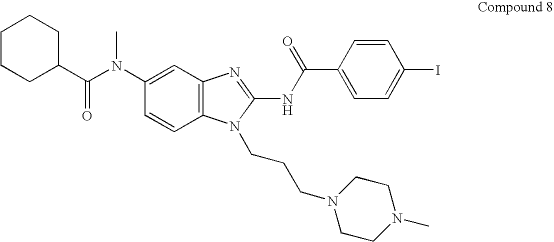

### Example 4

### ITK/KD/G354-Compound 4 X-Ray Diffraction Data Measurement

X-ray diffraction data were measured on ITK/KD/G354-Compound 4 crystals maintained at cryogenic temperature, typically at a value of about −160° C. X-ray diffraction data were measured with X-rays of wavelength 1.0061 Å at the PX6S beamline of the Swiss Light Source synchrotron using a MAR CCD 165 detector. Data were reduced to integrated intensities using the software D*TREK (Rigaku MSC) and to amplitudes using TRUNCATE (CCP4, 1994, Acta Crystallogr. D 50:760-763).

The crystals have a hexagonal unit cell whereby unit cell parameters are limited to a=b≠c and alpha=beta=90° and gamma=120°. The crystal space group symmetry is P6₄ and has unit cell dimensions a=b=239.68 Å, c=97.12 Å. These unit cell parameters commonly vary by 1 to 2% between samples.

### Example 5

### Structure Determination of the ITK/KD/G354-Compound 4 Complex

The crystal structure of the ITK/KD/G354-Compound 4 complex is determined by molecular replacement method. The structure of a homologous protein, Bruton's Tyrosine Kinase (BTK) catalytic domain (Mao et al., 2001, Journal of Biological Chemistry 276:41435-41443) is used as a template to solve the ITK/KD/G354-Compound 4 crystal structure. BTK atomic coordinates used are publicly available under entry 1K2P at the Protein Databank (Bernstein et al., 1977, Journal of Molecular Biology 112:535-542). An ITK/KD/G354 homology model is prepared with BTK residues A397 to A654 where equivalent ITK/KD/G354 residues are modeled in with arbitrary conformations. An initial ITK/KD/G354 protein structure is determined by molecular replacement (Rossmann, 1972, The Molecular Replacement Method, Gordon and Breach, New York) using the program AMORE (Navaza, 1994, Acta Crystallogr. A 50:157-163) as implemented in the CCP4 software suite (CCP4, 1994, Acta Crystallogr. D 50:760-763). The ITK/KD/G354 crystal molecular packing contains six protein molecules per asymmetric unit plus an estimated solvent content of 72%. Compound structures are built using Quanta and 0, and incorporated with the refinement. Two molecules of Compound 4 and two molecules of Compound 8 are clearly distinguishable and thus included in the refinement. The structure is then refined using the program CNX and is interspaced with model building using the software 0 (Jones et al., 1991, Acta Crystallogr. A 47:110-119). Refinement statistics are summarized in Table 4. The atomic coordinates are presented in Table 9.

TABLE 4
Statistics of Crystallographic Data and Structure Refinement

| Data collection | | |
|---|---|---|
| Space group | P6₄ | |
| Molecules per asymmetric unit | 6 | |
| Unit cell parameters a = b (Å), c (Å) | 239.68, 97.12 | |
| Average mosaicity (°) | 0.42 | |
| | all | outer shell |
| Resolution (Å) | 45.8 to 3.0 | 3.11 to 3.00 |
| Observed measurements | 364030 | 36179 |
| Unique reflections | 63877 | 6324 |
| Completeness (%) | 98.2 | 98.9 |
| Average I/σ_{I} | 7.8 | 2.2 |
| R_{sym}^{a} | 0.122 | 0.419 |
| Refinement | | |
| Resolution (Å) | 45.8 to 3.0 | |
| R_{factor}^{b}, reflections used | 0.31, 58143 | |
| Free R-value^{c}, reflections used | 0.37, 3092 | |
| R.m.s.d.^{d} bond lengths (Å) | 0.010 | |
| R.m.s.d. bond angles (°) | 1.48 | |

^{a}R_{sym} = Σ|Iᵢ − <I>|/ΣIᵢ where Iᵢ is the scaled intensity of the ith measurement and <I> is the mean intensity for that reflection.
^{b}R_{factor} = Σ|F_{obs} − F_{calc}|/Σ|F_{obs}| where F_{obs} and F_{calc} are the observed and calculated structure factor amplitudes, respectively.
^{c}Free R-value is the R_{factor} calculated from a set of reflections that are never used with the refinement. These reflections are used as a control set to pursue the refinement progress.
^{d}R.m.s.d. is the root mean square deviation from ideal geometry. Standard stereochemical parameters (Engh and Huber, 1991, Acta Crystallogr. A 47:392-400) were used with the refinement.

### Example 6

### Similarity Between the Kinase Domains of ITK and Other Kinases

To establish a measurement for the similarity between the kinase domains of ITK and other kinases, the following experiments were performed. Four sets of residues (RES-SET1, RES-SET2, RES-SET3, and RES-SET4) were identified by visual inspection of the ITK/KD/G354-Compound 4 structure as both being spatially proximal to the ATP binding site and as comprising distinct combinations of subregions within this site. These residue sets are defined in Table 5. The first set of residues (RES-SET1) comprises those residues which are located in the ATP binding site of ITK (i.e., V377, A389, K391, V419, I433, F435, E436, F437, M438, E439, H440, G441, C442, R486, L489); those residues which form the surface-exposed hydrophobic pocket (i.e., Q367, I369, L379, K387) into which the cyclohexyl moiety of Compound 4 binds; and those residues which are located in the G-loop (i.e., G370, S371), near the DFG-motif (i.e., S499, D500, F501), and near the kinase specificity pocket (i.e., A406, M410). The second set of residues (RES-SET2) comprises those residues which are located in the ATP binding site of ITK; and those residues which are located in the G-loop, near the DFG-motif, and near the kinase specificity pocket, as previously defined for the RES-SET1 residue set. The third set of residues (RES-SET3) comprises those residues which are located in the ATP binding site of ITK, as previously defined for the RES-SET1 residue set. The fourth set of residues (RES-SET4) comprises those residues which are located in the ATP binding site of ITK and those residues which form the surface-exposed hydrophobic pocket, as previously defined for the RES-SET1 residue set.

TABLE 5
Definition of residue sets.

| | |
|---|---|
| RES-SET1: | V377, A389, K391, V419, L433, F435, E436, F437, M438, |
| | E439, H440, G441, C442, R486, L489, G370, S371, E406, |
| | M410, S499, D500, F501, Q367, I369, L379, K387 |
| RES-SET2: | V377, A389, K391, V419, L433, F435, E436, F437, M438, |
| | E439, H440, G441, C442, R486, L489, G370, S371, E406, |
| | M410, S499, D500, F501 |
| RES-SET3: | V377, A389, K391, V419, L433, F435, E436, F437, M438, |
| | E439, H440, G441, C442, R486, L489 |
| RES-SET4: | V377, A389, K391, V419, L433, F435, E436, F437, M438, |
| | E439, H440, G441, C442, R486, L489, Q367, I369, L379, |
| | K387 |

The residue numbering in Table 5 is based on the position of that residue in full-length human ITK kinase. An alignment of the human ITK kinase domain with other known human kinase domain sequences was performed. The parts of this alignment that pertain to the aforedescribed 4 residue sets are presented in Table 10. Based on these partial sequence alignments, the sequence identities over these four residue sets were calculated between human ITK and each of the other presented human kinases. Table 6 lists, for each residue set, the kinase with the highest identity to human ITK and the associated percent identity, the kinase with the highest identity for which a structure is available and the associated percent identity, and the percent identities to human BTK, TXK, EGFR, and LCK kinases.

TABLE 6
Percent sequence identities^{a} between human ITK and other human Kinases

| | | Kinase with | | | | |
|---|---|---|---|---|---|---|
| | | highest | | | | |
| | Kinase with | similarity | | | | |
| | highest | and available | | | | |
| | similarity | structure | BTK | TXK | EGFR | LCK |
| RES-SET1 | 85 TXK | 73 CSK | 66 | 85 | 62 | 58 |
| RES-SET2 | 87 TEC | 73 BTK | 73 | 87 | 69 | 64 |
| RES-SET3 | 80 TEC | 67 BTK | 67 | 80 | 60 | 60 |
| RES-SET4 | 79 TXK | 69 CSK | 58 | 79 | 53 | 3 |

^{a}Based on the partial sequence alignments presented in Table 10

As a second measurement of similarity between the kinase domain of human ITK and that of other select human kinases, the backbone RMSDs have been calculated for ITK vs. BTK, ITK vs. EGFR, and ITK vs. LCK. Also the backbone RMSDs were calculated between the A and the B chain of the BTK structure to probe for the variability within a protein structure. The structures were taken from the Protein Databank entries 1K2P (BTK), 1M17 (EGFR), and 1QPJ (LCK). These crystal structures have respective resolutions of 2.1 Å, 2.2 Å, and 2.6 Å. The RMSDs are calculated by separately performing an alignment of the backbone atoms for the residues in each of the four residue sets and then measuring the RMSDs. This was done using the software program INSIGHT (Accelrys).

TABLE 7
Backbone RMSDs for the four residue sets

| | | | | BTK A vs |
|---|---|---|---|---|
| | ITK vs BTK | ITK vs EGFR | ITK vs LCK | BTK B |
| RES-SET1 | 1.80 | 1.09 | 0.79 | 1.17 |
| RES-SET2 | 1.75 | 1.13 | 0.74 | 1.14 |
| RES-SET3 | 1.08 | 0.61 | 0.60 | 0.74 |
| RES-SET4 | 0.57 | 0.50 | 0.47 | 0.32 |

The alignment for the RMSD measurements differed from that used in the sequence identity analysis in that K387 of ITK was aligned with D426 of BTK, or with P717 of EGFR. All other residues were aligned in the same way. The polypeptide binding pocket consisting of residues Q367, I369, L379, K387, and F437 of SEQ ID NO. 1 has a backbone-atom R.m.s.d. of 0.42 Angstroms to the corresponding residues in LCK, 0.55 Angstroms to the corresponding residues in EGFR, and 1.42 Angstroms to the corresponding residues in BTK. The RMSD is measured as described above.

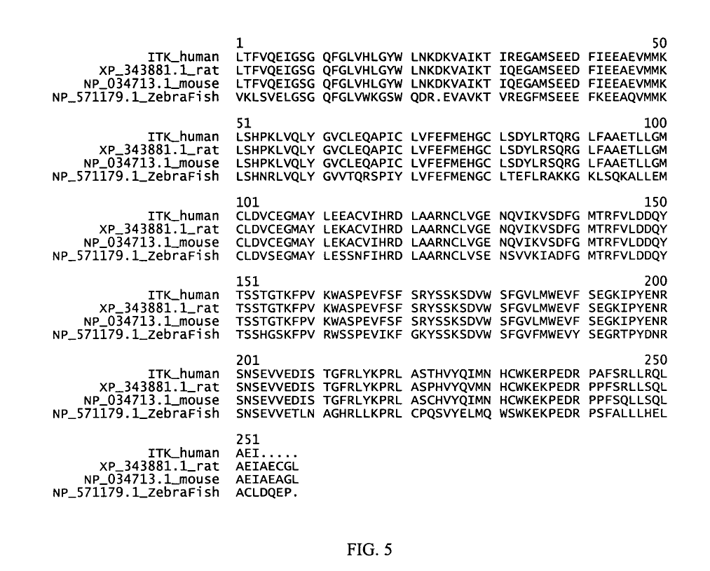

A third analysis of similarity was performed between the kinase domain of human ITK and that of the 3 known ITK orthologs. The sequences for human, rat, mouse, and zebrafish were aligned as shown in FIG. 5. The sequence similarities were then calculated for the four residue sets and are presented in see Table 9.

known ITK ortholog

| | ITK rat | ITK mouse | ITK zebrafish |
|---|---|---|---|
| RES-SET1 | 100 | 100 | 77 |
| RES-SET2 | 100 | 100 | 91 |
| RES-SET3 | 100 | 100 | 93 |
| RES-SET4 | 100 | 100 | 74 |

### Example 7

### Murine ITK/KD/G353-ITK Inhibitor Complex Formation, Crystallization and X-Ray Diffraction

A solution of murine ITK/KD/G353 protein (SEQ ID NO. 16) at a concentration of 10 to 31.5 mg/mL, previously frozen at −80° C. is thawed on ice for 30 minutes before use. A suitable compound which inhibits ITK, is dissolved with dimethyl sulfoxide (DMSO) to a concentration of 100 mg/mL at room temperature. The ITK inhibitor is mixed with the protein solution at 5 molar ratio of inhibitor to protein. The mixture is incubated on ice for 60 to 90 minutes.

Protein crystals are obtained by vapor diffusion methos using hanging drops at room temperature. A drop containing ITK/KD/G353-ITK inhibitor complex is let to equilibrate against a reservoir solution in a sealed container. In the present example, a hanging drop is prepared by mixing 1 μL of the ITK/KD/G353-ITK inhibitor with an equal volume of reservoir solution. The reservoir solution contains 10 to 18% polyethyleneglycol 5000 monomethylether, 100 mM sodium citrate pH 5 to 6 and 1 mM TCEP.

An ITK/KD/G353-ITK inhibitor crystal is transferred to a stabilizing solution containing 20% polyethyleneglycol 5000 monomethylether, 100 mM sodium citrate pH 5.5, 1 mM TCEP, 25% glycerol and 20 mM ITK inhibitor. The crystal is flash-frozen in a cold stream at cryogenic temperature of about −160° C. X-ray diffraction data are measured with an RU-H3R rotating anode-based generator (Rigaku/MSC) operating at 50 kV/60 mA equipped with an R-Axis-IV++ detector (Rigaku/MSC) and confocal blue optics (Osmics). X-ray diffraction data to 4 Å resolution are reduced with a combination of DENZO and SCALEPACK (Otwinowski and Minor, 1997, Meth. Enzymol. 276:307-326) and TRUNCATE (CCP4, 1994, Acta Crystallogr. D 50:760-763).

The crystal has a tetragonal unit cell whereby unit cell parameters are limited to a=b≠c and alpha=beta=gamma=90°. The crystal space group symmetry is P4₃2₁2 and has unit cell dimensions a=b=98.780 Å, c=122.408 Å. These unit cell parameters commonly vary by 1 to 2% between samples. The crystal has an estimated solvent content of about 75% and one protein assembly per asymmetric unit.

LENGTHY TABLE REFERENCED HERE
US20070032403A1-20070208-T00001
Please refer to the end of the specification for access instructions.

LENGTHY TABLE REFERENCED HERE
US20070032403A1-20070208-T00002
Please refer to the end of the specification for access instructions.

LENGTHY TABLE
The patent application contains a lengthy table section. A copy of the table is available in electronic form from the USPTO web site (http://seqdata.uspto.gov/?pageRequest=docDetail&DocID=US20070032403A1) An electronic copy of the table will also be available from the USPTO upon request and payment of the fee set forth in 37 CFR 1.19(b)(3).

What is claimed is:

1. A crystalline composition comprising an ITK kinase domain-ligand complex.

2. The crystalline composition according to claim 1 wherein the ITK kinase domain is human.

3. The crystalline composition according to claim 2, wherein said composition effectively diffracts X-rays such that the atomic coordinates of the ITK kinase domain-ligand complex can be determined to a resolution of better than 5.0 Angstroms.

4. The crystalline composition according to claim 2, wherein said composition effectively diffracts X-rays such that the atomic coordinates of the ITK kinase domain-ligand complex can be determined to a resolution of 3.0 Angstroms or better.

5. The crystalline composition according to claims 2, 3 or 4 wherein the ITK kinase domain is chosen from ITK/KD/Q343 (SEQ ID NO. 9), ITK/KD/G354 (SEQ ID NO. 1) and ITK/KD/S361 (SEQ ID NO. 12).

6. The crystalline composition according to claims 2, 3 or 4 wherein the ITK kinase domain is ITK/KD/G354 (SEQ ID NO. 1).

7. The crystalline composition according to claim 1 wherein the crystals have a hexagonal unit cell whereby unit cell parameters are limited to a=b≠c and alpha=beta=90° and gamma=120°, and the crystal space group symmetry is P6₄ and has unit cell dimensions a=b=239.68 Å, c=97.12 Å wherein the unit cell parameters can vary by 1 to 2%.

8. The crystalline composition according to any of claims 1-7, wherein said ITK kinase domain-ligand complex has a three-dimensional structure comprising the atomic coordinates as defined in Table 9.

9. The crystalline composition according to any of claims 1-7, wherein the ligand is one chosen from Table 2.

10. An isolated polypeptide binding pocket comprising the homologous amino acid residues, based on the kinase-domain residue alignments presented in Table 10, to Q367, I369, L379, K387, and F437 of SEQ ID NO. 1, numbered based on the position in the full-length, wild-type human ITK kinase wherein the backbone-atom R.m.s.d is less than 0.42 Angstroms from the coordinates given in Table 9.

11. An isolated polypeptide binding pocket wherein the isolated polypeptide binding pocket comprises amino acid residues Q367, I369, L379, K387, and F437 of SEQ ID NO. 1, numbered based on the position in the full-length, wild-type human ITK kinase wherein the backbone-atom R.m.s.d is less than 3 Angstroms from the coordinates given in Table 9.

12. An isolated polypeptide binding pocket comprising the homologous amino acid residues, based on the kinase-domain residue alignments presented in Table 10, to V377, A389, K391, V419, L433, F435, E436, F437, M438, E439, H440, G441, C442, R486, L489, G370, S371, E406, M410, S499, D500, F501, Q367, I369, L379, K387 (RES-SET1) of SEQ ID NO. 1, numbered based on the position in the full-length, wild-type human ITK kinase wherein the backbone-atom R.m.s.d is less than 0.79 Angstroms from the coordinates given in Table 9.

13. An isolated polypeptide binding pocket wherein the isolated polypeptide binding pocket comprises amino acid residues V377, A389, K391, V419, L433, F435, E436, F437, M438, E439, H440, G441, C442, R486, L489, G370, S371, E406, M410, S499, D500, F501, Q367, I369, L379, K387 (RES-SET1) of SEQ ID NO. 1, numbered based on the position in the full-length, wild-type human ITK kinase wherein the backbone-atom R.m.s.d is less than 3 Angstroms from the coordinates given in Table 9.

14. An isolated polypeptide binding pocket comprising the homologous amino acid residues, based on the kinase-domain residue alignments presented in Table 10, to V377, A389, K391, V419, L433, F435, E436, F437, M438, E439, H440, G441, C442, R486, L489, G370, S371, E406, M410, S499, D500, F501 (RES-SET2) of SEQ ID NO. 1, numbered based on the position in the full-length, wild-type human ITK kinase wherein the backbone-atom R.m.s.d is less than 0.74 Angstroms from the coordinates given in Table 9.

15. An isolated polypeptide binding pocket wherein the isolated polypeptide binding pocket comprises amino acid residues V377, A389, K391, V419, L433, F435, E436, F437, M438, E439, H440, G441, C442, R486, L489, G370, S371, E406, M410, S499, D500, F501 (RES-SET2) of SEQ ID NO. 1, numbered based on the position in the full-length, wild-type human ITK kinase wherein the backbone-atom R.m.s.d is less than 3 Angstroms from the coordinates given in Table 9.

16. An isolated polypeptide binding pocket comprising the homologous amino acid residues, based on the kinase-domain residue alignments presented in Table 10, to V377, A389, K391, V419, L433, F435, E436, F437, M438, E439, H440, G441, C442, R486, L489 (RES-SET3) of SEQ ID NO. 1, numbered based on the position in the full-length, wild-type human ITK kinase wherein the backbone-atom R.m.s.d is less than 0.60 Angstroms from the coordinates given in Table 9.

17. An isolated polypeptide binding pocket wherein the isolated polypeptide binding pocket comprises amino acid residues V377, A389, K391, V419, L433, F435, E436, F437, M438, E439, H440, G441, C442, R486, L489 (RES-SET3) of SEQ ID NO. 1, numbered based on the position in the full-length, wild-type human ITK kinase wherein the backbone-atom R.m.s.d is less than 3 Angstroms from the coordinates given in Table 9.

18. An isolated polypeptide binding pocket comprising the homologous amino acid residues, based on the kinase-domain residue alignments presented in Table 10, to V377, A389, K391, V419, L433, F435, E436, F437, M438, E439, H440, G441, C442, R486, L489, Q367, I369, L379, K387 (RES-SET4) of SEQ ID NO. 1, numbered based on the position in the full-length, wild-type human ITK kinase wherein the backbone-atom R.m.s.d is less than 0.47 Angstroms from the coordinates given in Table 9.

19. An isolated polypeptide binding pocket wherein the isolated polypeptide binding pocket comprises amino acid residues V377, A389, K391, V419, L433, F435, E436, F437, M438, E439, H440, G441, C442, R486, L489, Q367, I369, L379, K387 (RES-SET4) of SEQ ID NO. 1, numbered based on the position in the full-length, wild-type human ITK kinase wherein the backbone-atom R.m.s.d is less than 3 Angstroms from the coordinates given in Table 9.

20. The isolated polypeptide binding pocket according to any one of claims 10-19 wherein the ligand is one chosen from Table 2.

21. The binding pocket according to claim 10, wherein F437 of said binding pocket interacts with a ligand, said interaction resulting in a distance of about 3.8 Angstroms between F437 and the ligand.

22. A method of obtaining crystals of the ITK kinase domain protein of SEQ ID NO. 1 in a complex with a ligand, said process comprising:
(a) obtaining a crystallizable composition, with said crystallizable composition comprising an ITK kinase domain protein, cations and a ligand; and
(b) subjecting the composition of step (a) to conditions which promote crystallization.

23. The process according to claim 22, wherein the ligand is one chosen from Table 2.

24. A method of identifying an ITK inhibitor, said method comprising:
identifying a compound as a potential inhibitor by performing rational drug design with a three-dimensional structure determined for the crystalline composition according to claim 1;
synthesizing the compound;
determining whether the compound inhibits the activity of ITK.

25. The method according to claim 24, wherein the rational drug design is performed in conjunction with computer modeling.

26. A method of identifying an ITK inhibitor, said method comprising:
contacting a compound with a binding pocket according to claim 10;
determining whether the compound inhibits the activity of ITK.

27. A computer assisted method for identifying an inhibitor of ITK activity comprising:
supplying a computer modeling application with a set of coordinates of the binding pocket according to any one of claims 10-19;
supplying a computer modeling application with a set of coordinates of one or more chemical entities;
scoring the binding modes for said one or more chemical entities; and
selecting an inhibitor based on the assigned binding mode scores.

28. The method according to claim 27 wherein the computer modeling application is further supplied with a set of coordinates in Table 9.

29. A method of growing crystals comprising
providing a solution of SEQ ID NO. 1 polypeptide complexed with a ligand;
providing a precipitant solution; and
combining the precipitant and solution of SEQ ID NO. 1 polypeptide complexed with a ligand; and
allowing crystals of SEQ ID NO. 1 polypeptide complexed with a ligand to form.

30. The method according to claim 29 wherein the precipitant is 5 to 15% polyethylene glycol 1500 (PEG 1500) and is provided by vapor diffusion.

31. The method according to claim 29 wherein the precipitant is 8 to 12% polyethylene glycol 1500 (PEG 1500).

32. An isolated polypeptide chosen from SEQ ID NO. 1, SEQ ID NO. 9, SEQ ID NO. 10 and SEQ ID NO. 12.

33. A method of determining the three-dimensional structure of a complex comprising one or more ligands and an ITK kinase domain, comprising:
a) crystallizing the ITK kinase domain-ligand complex;
b) obtaining X-ray diffraction data for the crystals of said ITK kinase domain-ligand complex;
c) using the atomic coordinates of Table 9 to determine the three dimensional structure of the ITK kinase domain-ligand complex.

34. An isolated polypeptide chosen from SEQ ID NO. 13, SEQ ID NO. 14, SEQ ID NO. 15 and SEQ ID NO. 16.

35. An isolated polypeptide binding pocket comprising amino acid residues F435, M438, and C442 of SEQ ID NO. 1, numbered based on the position in the full-length, wild-type human ITK kinase wherein the backbone-atom R.m.s.d is less than 3 Angstroms from the coordinates given in Table 9.

36. The binding pocket according to claim 35, wherein F435, M438, and C442 of said binding pocket interacts with a ligand, said interaction resulting in a distance of about 4 Angstroms between the ligand and the aromatic side chain of F435, of about 2.7 Angstroms between the ligand and the backbone nitrogen atom of M438, of about 3.1 Angstroms between the ligand and the backbone carbonyl oxygen of M438, and of about 2.7 Angstroms between the ligand and the side chain sulfur atom of C442.

37. An isolated polypeptide binding pocket comprising amino acid residues F435 and M438 of SEQ ID NO. 1, numbered based on the position in the full length, wild type human ITK kinase, wherein F435 and M438 of said binding pocket interacts with a ligand, said interaction resulting in a distance of about 4 Angstroms between the ligand and the aromatic side chain of F435, of about 2.7 Angstroms between the ligand and the backbone nitrogen atom of M438, and of about 3.1 Angstroms between the ligand and the backbone carbonyl oxygen of M438.


SEQUENCE LISTING (16 sequences)

SEQ ID NO: 1 - protein, 270 aa, Homo sapiens
GSMGKWVIDPSELTFVQEIGSGQFGLVHLGYWLNKDKVAIKTIREGAMSEEDFIEEAEVMMKLSHPKLVQLYGVCLEQAPICLVFEFMEHGCLSDYLRTQRGLFAAETLLGMCLDVCEGMAYLEEACVIHRDLAARNCLVGENQVIKVSDFGMTRFVLDDQYTSSTGTKFPVKWASPEVFSFSRYSSKSDVWSFGVLMWEVFSEGKIPYENRSNSEVVEDISTGFRLYKPRLASTHVYQIMNHCWKERPEDRPAFSRLLRQLAEIAESGL

SEQ ID NO: 2 - protein, 620 aa, Homo sapiens
MNNFILLEEQLIKKSQQKRRTSPSNFKVRFFVLTKASLAYFEDRHGKKRTLKGSIELSRIKCVEIVKSDISIPCHYKYPFQVVHDNYLLYVFAPDRESRQRWVLALKEETRNNNSLVPKYHPNFWMDGKWRCCSQLEKLATGCAQYDPTKNASKKPLPPTPEDNRRPLWEPEETVVIALYDYQTNDPQELALRRNEEYCLLDSSEIHWWRVQDRNGHEGYVPSSYLVEKSPNNLETYEWYNKSISRDKAEKLLLDTGKEGAFMVRDSRTAGTYTVSVFTKAVVSENNPCIKHYHIKETNDNPKRYYVAEKYVFDSIPLLINYHQHNGGGLVTRLRYPVCFGRQKAPVTAGLRYGKWVIDPSELTFVQEIGSGQFGLVHLGYWLNKDKVAIKTIREGAMSEEDFIEEAEVMMKLSHPKLVQLYGVCLEQAPICLVFEFMEHGCLSDYLRTQRGLFAAETLLGMCLDVCEGMAYLEEACVIHRDLAARNCLVGENQVIKVSDFGMTRFVLDDQYTSSTGTKFPVKWASPEVFSFSRYSSKSDVWSFGVLMWEVFSEGKIPYENRSNSEVVEDISTGFRLYKPRLASTHVYQIMNHCWKERPEDRPAFSRLLRQLAEIAESGL

SEQ ID NO: 3 - protein, 279 aa, Homo sapiens
MQKAPVTAGLRYGKWVIDPSELTFVQEIGSGQFGLVHLGYWLNKDKVAIKTIREGAMSEEDFIEEAEVMMKLSHPKLVQLYGVCLEQAPICLVFEFMEHGCLSDYLRTQRGLFAAETLLGMCLDVCEGMAYLEEACVIHRDLAARNCLVGENQVIKVSDFGMTRFVLDDQYTSSTGTKFPVKWASPEVFSFSRYSSKSDVWSFGVLMWEVFSEGKIPYENRSNSEVVEDISTGFRLYKPRLASTHVYQIMNHCWKERPEDRPAFSRLLRQLAEIAESGL

SEQ ID NO: 4 - DNA, 4883 bp, Viral
gggcgaattgggcccgacgtcgcatgctcccggccgccatggccgcgggattgggatccatgaacaactttatcctcctggaagaacagctcatcaagaaatcccaacaaaagagaagaacttctccctcgaactttaaagtccgcttctttgtgttaaccaaagccagcctggcatactttgaagatcgtcatgggaagaagcgcacgctgaaggggtccattgagctctcccgaatcaaatgtgttgagattgtgaaaagtgacatcagcatcccatgccactataaatacccgtttcaggtggtgcatgacaactacctcctatatgtgtttgctccagatcgtgagagccggcagcgctgggtgctggcccttaaagaagaaacgaggaataataacagtttggtgcctaaatatcatcctaatttctggatggatgggaagtggaggtgctgttctcagctggagaagcttgcaacaggctgtgcccaatatgatccaaccaagaatgcttcaaagaagcctcttcctcctactcctgaagacaacaggcgaccactttgggaacctgaagaaactgtggtcattgccttatatgactaccaaaccaatgatcctcaggaactcgcactgcggcgcaacgaagagtactgcctgctggacagttctgagattcactggtggagagtccaggacaggaatgggcatgaaggatatgtaccaagcagttatctggtggaaaaatctccaaataatctggaaacctatgagtggtacaataagagtatcagccgagacaaagctgaaaaacttcttttggacacaggcaaagaaggagccttcatggtaagggattccaggactgcaggaacatacaccgtgtctgttttcaccaaggctgttgtaagtgagaacaatccctgtataaagcattatcacatcaaggaaacaaatgacaatcctaagcgatactatgtggctgaaaagtatgtgttcgattccatccctcttctcatcaactatcaccaacataatggaggaggcctggtgactcgactccggtatccagtttgttttgggaggcagaaagccccagttacagcagggctgagatacgggaaatgggtgatcgacccctcagagctcacttttgtgcaagagattggcagtgggcaatttgggttggtgcatctgggctactggctcaacaaggacaaggtggctatcaaaaccattcgggaaggggctatgtcagaagaggacttcatagaggaggctgaagtaatgatgaaactctctcatcccaaactggtgcagctgtatggggtgtgcctggagcaggcccccatctgcctggtgtttgagttcatggagcacggctgcctgtcagattatctacgcacccagcggggactttttgctgcagagaccctgctgggcatgtgtctggatgtgtgtgagggcatggcctacctggaagaggcatgtgtcatccacagagacttggctgccagaaattgtttggtgggagaaaaccaagtcatcaaggtgtctgactttgggatgacaaggttcgttctggatgatcagtacaccagttccacaggcaccaaattcccggtgaagtgggcatccccagaggttttctctttcagtcgctatagcagcaagtccgatgtgtggtcatttggtgtgctgatgtgggaagttttcagtgaaggcaaaatcccgtatgaaaaccgaagcaactcagaggtggtggaagacatcagtaccggatttcggttgtacaagccccggctggcctccacacacgtctaccagattatgaatcactgctggaaagagagaccagaagatcggccagccttctccagactgctgcgtcaactggctgaaattgcagaatcaggactttaggcggccgcaatcactagtgcggccgcctgcaggtcgaccatatgggagagctcccaacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttcgataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaataccgcgcccggcgaccgagttgctcttgcccggcgtcaatacgggataatagtgtatgacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgtatgcggtgtgaaataccgcacagatgcgtaaggagaaaataccgcatcaggcgaaattgtaaacgttaatattttgttaaaattcgcgttaaatatttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactata

SEQ ID NO: 5 - protein, 505 aa, Homo sapiens
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSMQKAPVTAGLRYGKWVIDPSELTFVQEIGSGQFGLVHLGYWLNKDKVAIKTIREGAMSEEDFIEEAEVMMKLSHPKLVQLYGVCLEQAPICLVFEFMEHGCLSDYLRTQRGLFAAETLLGMCLDVCEGMAYLEEACVIHRDLAARNCLVGENQVIKVSDFGMTRFVLDDQYTSSTGTKFPVKWASPEVFSFSRYSSKSDVWSFGVLMWEVFSEGKIPYENRSNSEVVEDISTGFRLYKPRLASTHVYQIMNHCWKERPEDRPAFSRLLRQLAEIAESGL

SEQ ID NO: 6 - protein, 268 aa, Homo sapiens
MGKWVIDPSELTFVQEIGSGQFGLVHLGYWLNKDKVAIKTIREGAMSEEDFIEEAEVMMKLSHPKLVQLYGVCLEQAPICLVFEFMEHGCLSDYLRTQRGLFAAETLLGMCLDVCEGMAYLEEACVIHRDLAARNCLVGENQVIKVSDFGMTRFVLDDQYTSSTGTKFPVKWASPEVFSFSRYSSKSDVWSFGVLMWEVFSEGKIPYENRSNSEVVEDISTGFRLYKPRLASTHVYQIMNHCWKERPEDRPAFSRLLRQLAEIAESGL

SEQ ID NO: 7 - protein, 494 aa, Homo sapiens
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSMGKWVIDPSELTFVQEIGSGQFGLVHLGYWLNKDKVAIKTIREGAMSEEDFIEEAEVMMKLSHPKLVQLYGVCLEQAPICLVFEFMEHGCLSDYLRTQRGLFAAETLLGMCLDVCEGMAYLEEACVIHRDLAARNCLVGENQVIKVSDFGMTRFVLDDQYTSSTGTKFPVKWASPEVFSFSRYSSKSDVWSFGVLMWEVFSEGKIPYENRSNSEVVEDISTGFRLYKPRLASTHVYQIMNHCWKERPEDRPAFSRLLRQLAEIAESGL

SEQ ID NO: 8 - protein, 494 aa, Homo sapiens
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSMGKWVIDPSELTFVQEIGSGQFGLVHLGYWLNKDKVAIKTIREGAMSEEDFIEEAEVMMKLSHPKLVQLYGVCLEQAPICLVFEYMEHGCLSDYLRTQRGLFAAETLLGMCLDVCEGMAYLEEACVIHRDLAARNCLVGENQVIKVSDFGMTRFVLDDQYTSSTGTKFPVKWASPEVFSFSRYSSKSDVWSFGVLMWEVFSEGKIPYENRSNSEVVEDISTGFRLYKPRLASTHVYQIMNHCWKERPEDRPAFSRLLRQLAEIAESGL

SEQ ID NO: 9 - protein, 281 aa, Homo sapiens
GSMQKAPVTAGLRYGKWVIDPSELTFVQEIGSGQFGLVHLGYWLNKDKVAIKTIREGAMSEEDFIEEAEVMMKLSHPKLVQLYGVCLEQAPICLVFEFMEHGCLSDYLRTQRGLFAAETLLGMCLDVCEGMAYLEEACVIHRDLAARNCLVGENQVIKVSDFGMTRFVLDDQYTSSTGTKFPVKWASPEVFSFSRYSSKSDVWSFGVLMWEVFSEGKIPYENRSNSEVVEDISTGFRLYKPRLASTHVYQIMNHCWKERPEDRPAFSRLLRQLAEIAESGL

SEQ ID NO: 10 - protein, 261 aa, Homo sapiens
MSELTFVQEIGSGQFGLVHLGYWLNKDKVAIKTIREGAMSEEDFIEEAEVMMKLSHPKLVQLYGVCLEQAPICLVFEFMEHGCLSDYLRTQRGLFAAETLLGMCLDVCEGMAYLEEACVIHRDLAARNCLVGENQVIKVSDFGMTRFVLDDQYTSSTGTKFPVKWASPEVFSFSRYSSKSDVWSFGVLMWEVFSEGKIPYENRSNSEVVEDISTGFRLYKPRLASTHVYQIMNHCWKERPEDRPAFSRLLRQLAEIAESGL

SEQ ID NO: 11 - protein, 487 aa, Homo sapiens
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSMSELTFVQEIGSGQFGLVHLGYWLNKDKVAIKTIREGAMSEEDFIEEAEVMMKLSHPKLVQLYGVCLEQAPICLVFEFMEHGCLSDYLRTQRGLFAAETLLGMCLDVCEGMAYLEEACVIHRDLAARNCLVGENQVIKVSDFGMTRFVLDDQYTSSTGTKFPVKWASPEVFSFSRYSSKSDVWSFGVLMWEVFSEGKIPYENRSNSEVVEDISTGFRLYKPRLASTHVYQIMNHCWKERPEDRPAFSRLLRQLAEIAESGL

SEQ ID NO: 12 - protein, 263 aa, Homo sapiens
GSMSELTFVQEIGSGQFGLVHLGYWLNKDKVAIKTIREGAMSEEDFIEEAEVMMKLSHPKLVQLYGVCLEQAPICLVFEFMEHGCLSDYLRTQRGLFAAETLLGMCLDVCEGMAYLEEACVIHRDLAARNCLVGENQVIKVSDFGMTRFVLDDQYTSSTGTKFPVKWASPEVFSFSRYSSKSDVWSFGVLMWEVFSEGKIPYENRSNSEVVEDISTGFRLYKPRLASTHVYQIMNHCWKERPEDRPAFSRLLRQLAEIAESGL

SEQ ID NO: 13 - protein, 619 aa, Murinae gen. sp.
MNNFILLEEQLIKKSQQKRRTSPSNFKVRFFVLTKASLAYFEDRHGKKRTLKGSIELSRIKCVEIVKSDISIPCHYKYPFQVVHDNYLLYVFAPDCESRQRWVLTLKEETRNNNSLVSKYHPNFWMDGRWRCCSQLEKPAVGCAPYDPSKNASKKPLPPTPEDNRRSFQEPEETLVIALYDYQTNDPQELALRCDEEYYLLDSSEIHWWRVQDKNGHEGYAPSSYLVEKSPNNLETYEWYNKSISRDKAEKLLLDTGKEGAFMVRDSRTPGTYTVSVFTKAIISENPCIKHYHIKETNDSPKRYYVAEKYVFDSIPLLIQYHQYNGGGLVTRLRYPVCSWRQKAPVTAGLRYGKWVIQPSELTFVQEIGSGQFGLVHLGYWLNKDKVAIKTIQEGAMSEEDFIEEAEVMMKLSHPKLVQLYGVCLEQAPICLVFEFMEHGCLSDYLRSQRGLFAAETLLGMCLDVCEGMAYLEKACVIHRDLAARNCLVGENQVIKVSDFGMTRFVLDDQYTSSTGTKFPVKWASPEVFSFSRYSSKSDVWSFGVLMWEVFSEGKIPYENRSNSEVVEDISTGFRLYKPRLASCHVYQIMNHCWKEKPEDRPPFSQLLSQLAEIAEAGL

SEQ ID NO: 14 - protein, 268 aa, Murinae gen. sp.
MGKWVIQPSELTFVQEIGSGQFGLVHLGYWLNKDKVAIKTIQEGAMSEEDFIEEAEVMMKLSHPKLVQLYGVCLEQAPICLVFEFMEHGCLSDYLRSQRGLFAAETLLGMCLDVCEGMAYLEKACVIHRDLAARNCLVGENQVIKVSDFGMTRFVLDDQYTSSTGTKFPVKWASPEVFSFSRYSSKSDVWSFGVLMWEVFSEGKIPYENRSNSEVVEDISTGFRLYKPRLASCHVYQIMNHCWKEKPEDRPPFSQLLSQLAEIAEAGL

SEQ ID NO: 15 - protein, 494 aa, Murinae gen. sp.
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSMGKWVIQPSELTFVQEIGSGQFGLVHLGYWLNKDKVAIKTIQEGAMSEEDFIEEAEVMMKLSHPKLVQLYGVCLEQAPICLVFEFMEHGCLSDYLRSQRGLFAAETLLGMCLDVCEGMAYLEKACVIHRDLAARNCLVGENQVIKVSDFGMTRFVLDDQYTSSTGTKFPVKWASPEVFSFSRYSSKSDVWSFGVLMWEVFSEGKIPYENRSNSEVVEDISTGFRLYKPRLASCHVYQIMNHCWKEKPEDRPPFSQLLSQLAEIAEAGL

SEQ ID NO: 16 - protein, 270 aa, Murinae gen. sp.
GSMGKWVIQPSELTFVQEIGSGQFGLVHLGYWLNKDKVAIKTIQEGAMSEEDFIEEAEVMMKLSHPKLVQLYGVCLEQAPICLVFEFMEHGCLSDYLRSQRGLFAAETLLGMCLDVCEGMAYLEKACVIHRDLAARNCLVGENQVIKVSDFGMTRFVLDDQYTSSTGTKFPVKWASPEVFSFSRYSSKSDVWSFGVLMWEVFSEGKIPYENRSNSEVVEDISTGFRLYKPRLASCHVYQIMNHCWKEKPEDRPPFSQLLSQLAEIAEAGL